(19) United States
(12) Patent Application Publication
(10) Pub. No.: US 2005/0019259 A1
(43) Pub. Date: Jan. 27, 2005

(54) Screening methods for pathogen virulence factors under low oxygen conditions

(76) Inventors: Frederick M. Ausubel, Newton, MA (US); Stephen B. Calderwood, Wellesley, MA (US); Eleftherios E. Mylonakis, Boston, MA (US)

Correspondence Address: CLARK & ELBING LLP, 101 FEDERAL STREET, BOSTON, MA, 02110, US

(21) Appl. No.: 10/477,859

(22) PCT Filed: May 16, 2002
(86) PCT No.: PCT/US02/15410

(30) Foreign Application Priority Data
May 16, 2001 (US) 60291486

(51) Int. Cl. A61K 49/00

(52) U.S. Cl. 424/9.2

(57) ABSTRACT

In general, the invention relates to screening methods for identifying pathogen virulence factors expressed under conditions of low oxygen and for identifying drugs that inhibit a pathogen. The method includes the steps of: (a) exposing a nematode to a mutagenized pathogen cultured under a low oxygen condition; (b) determining whether the mutant pathogen infects the nematode, a reduction of disease in the nematode relative to that caused by the non-mutagenized pathogen indicating a mutation in a pathogenic virulence factor; and (c) using the mutation as a marker for identifying the pathogenic virulence factor.

### BACKGROUND OF THE INVENTION

The invention relates to screening methods for identifying pathogen virulence factors expressed under conditions of low oxygen and for identifying drugs that inhibit a pathogen.

Microbial pathogens use a variety of complex strategies to subvert host cellular functions, ensuring their multiplication and survival. To this end, some pathogens require low oxygen conditions for their proliferation, and utilize finely tuned host-specific strategies to establish a pathogenic relationship. During infection, pathogens encounter different conditions, and respond by expressing virulence factors that are appropriate for the particular environment, host, or both.

Although antibiotics have been effective tools in treating infectious disease, the emergence of drug resistant pathogens is becoming problematic in the clinical setting. New antibiotic or antipathogenic molecules are therefore needed to combat such drug resistant pathogens. Accordingly, there is a need in the art for screening methods aimed not only at identifying and characterizing potential antipathogenic agents, but also for identifying and characterizing the virulence factors that enable pathogens to infect and debilitate their hosts, especially those virulence factors expressed under conditions of low oxygen.

### SUMMARY OF THE INVENTION

In general, the invention features a method for identifying a pathogenic virulence factor. The method includes the steps of: (a) exposing a nematode to a mutagenized pathogen cultured under a low oxygen condition; (b) determining whether the mutant pathogen infects the nematode, a reduction of disease in the nematode relative to that caused by the non-mutagenized pathogen indicating a mutation in a pathogenic virulence factor; and (c) using the mutation as a marker for identifying the pathogenic virulence factor. In preferred embodiments, the nematode (for example, Caenorhabditis elegans) and the mutant pathogen (for example, a member of the genus of Enterococcus, Bacteroides, Propionibacterium, or Clostridium) are cultured together under a low oxygen condition. In other preferred embodiments, the method utilizes a bacterial/C. elegans killing assay, wherein the bacterial pathogen causes less C. elegans killing than the non-mutagenized bacterial pathogen.

In another aspect, the invention features a method of identifying a compound that inhibits pathogenicity of a bacterial pathogen. The method includes the steps of: (a) providing a nematode including a pathogen cultured under a low oxygen condition; (b) contacting the nematode with a test compound; and (c) determining whether the test compound inhibits the pathogenicity of the pathogen in the nematode. In preferred embodiments, the nematode and the mutant pathogen are cultured together under a low oxygen condition. Preferably, the test compound is provided in a compound library; is a small organic compound; is an inorganic compound; or is a peptide, peptidomimetic, or antibody or fragment thereof. In other preferred embodiments, inhibition of pathogenicity is measured by a bacterial/C. elegans killing assay, wherein the bacterial pathogen causes less C. elegans killing in the presence of the test compound than in the absence of the test compound.

By “virulence factor” is meant a cellular component (for example, a protein such as a transcription factor or a molecule without which the pathogen is incapable of causing disease) that gives an advantage to a microorganism to cause disease or to colonize a host (for example, a eukaryotic host organism such as a nematode or mammal). Such cellular components are involved in the adaptation of the pathogen to a host (for example, a nematode host), establishment and maintenance of an infection, and generation of the damaging effects of the infection to the host organism. Further, the phrase includes cellular components that act directly on host tissue, as well as components that regulate the activity or production of other pathogenic factors.

By “infection” or “infected” is meant an invasion or colonization of a host animal (e.g., nematode) by a pathogen that is damaging to the host or to others.

By “pathogen” is meant a causative agent of disease. Exemplary pathogens include bacteria, fungi, protists, and viruses, especially pathogens that thrive under conditions of low oxygen. Such pathogens may also be in a variety of developmental forms or stages including, without limitation, spores and biofilms.

By “inhibits pathogenicity of a pathogen” is meant the ability of a test compound to decrease, suppress, attenuate, diminish, arrest, or stabilize the development or progression of a pathogen-mediated disease or infection in a eukaryotic host organism. Preferably, such inhibition decreases pathogenicity by at least 5%, more preferably by at least 25%, and most preferably by at least 50% or more, as compared to symptoms in the absence of the test compound in any appropriate pathogenicity assay (for example, those assays described herein). In one particular example, inhibition may be measured by monitoring pathogenic symptoms in a nematode infected with a pathogen exposed to a test compound or extract, a decrease in the level of pathogenic symptoms relative to the level of symptoms in the host organism not exposed to the compound indicating compound-mediated inhibition of the pathogen.

By “low oxygen conditions” or “under a condition of low oxygen” is meant an environment having less than twenty percent (20%) oxygen, preferably less than 15%, more preferably less than ten percent (10%), and most preferably less than five percent (5%) oxygen.

The present invention provides a number of advantages. For example, the invention facilitates the identification of novel targets and therapeutic approaches for identifying or preparing agents active on pathogenic virulence factors and genes expressed under conditions of low oxygen. The invention also provides long awaited advantages over a wide variety of standard screening methods used for distinguishing and evaluating the efficacy of a compound against a variety of pathogens that live and thrive under varying conditions of low oxygen. In one particular example, the screening methods described herein allow for the simultaneous evaluation of host toxicity as well as anti-pathogenic potency in a simple in vivo screen. Moreover, the methods of the invention allow one to evaluate the ability of a compound to inhibit pathogenesis and infection of a pathogen (for example, a bacterium), and, at the same time, to evaluate the ability of the compound to stimulate and strengthen a host's response to pathogenic attack.

Accordingly, the methods of the invention provide a straightforward means to identify compounds that are both safe for use in eukaryotic host organisms (i.e., compounds which do not adversely affect the normal development and physiology of the organism) and efficacious against a variety of pathogenic microbes. In addition, the methods of the invention provide a route for analyzing virtually any number of compounds for an anti-pathogenic effect with high-volume throughput, high sensitivity, and low complexity. The methods are also relatively inexpensive to perform and enable the analysis of small quantities of active substances found in either purified or crude extract form. Furthermore, the methods disclosed herein provide a means for identifying anti-pathogenic compounds that cross eukaryotic cell membranes and which maintain therapeutic efficacy in an in vivo method of administration. In addition, the above-described methods of screening are suitable for both known and unknown compounds and compound libraries, including synthetic combinatorial chemical libraries.

Other features and advantages of the invention will be apparent from the following description of the preferred embodiments thereof, and from the claims.

### DETAILED DESCRIPTION

The drawings will first be described.

Drawings

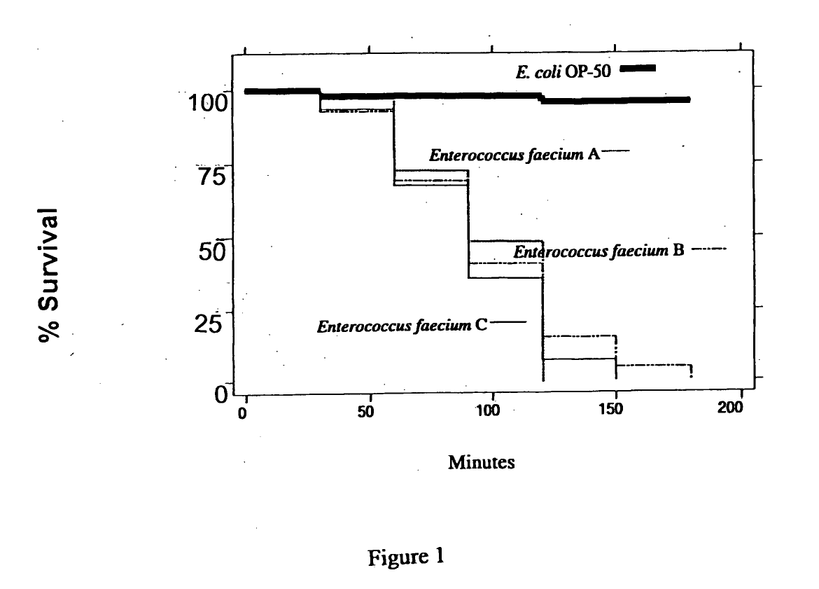

FIG. 1 shows the Kaplan-Meier survival estimates of killing of C. elegans by three different strains of Enterococcus faecium, A, B, and C, each cultured under low oxygen conditions, as compared to E. coli OP-50.

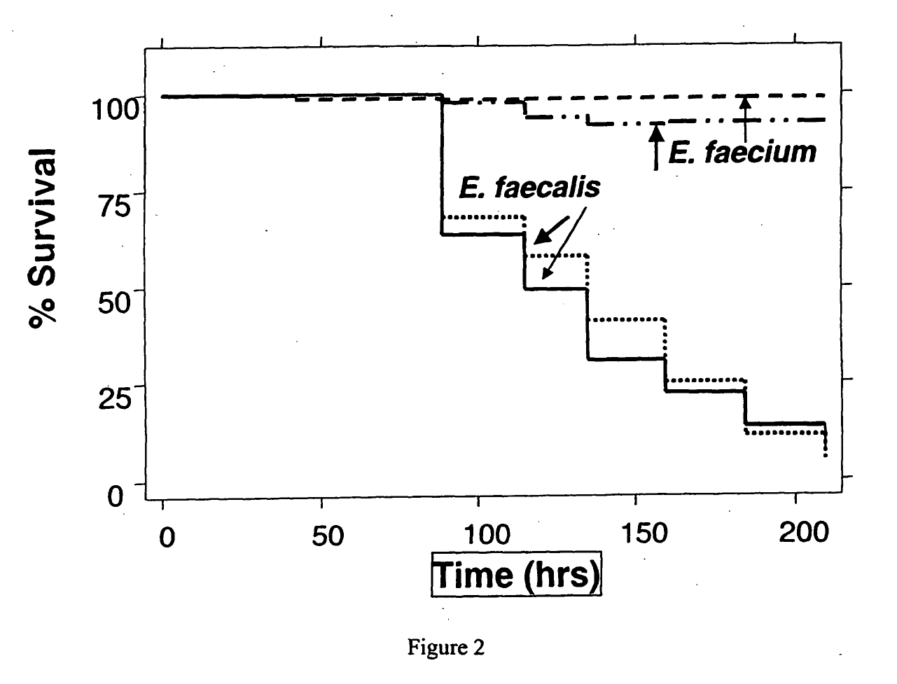

FIG. 2 shows the Kaplan-Meier survival estimates of C. elegans, under aerobic conditions, by Enterococcus faecium, cultured without exposure to conditions of low oxygen, and Enterococcus faecalis.

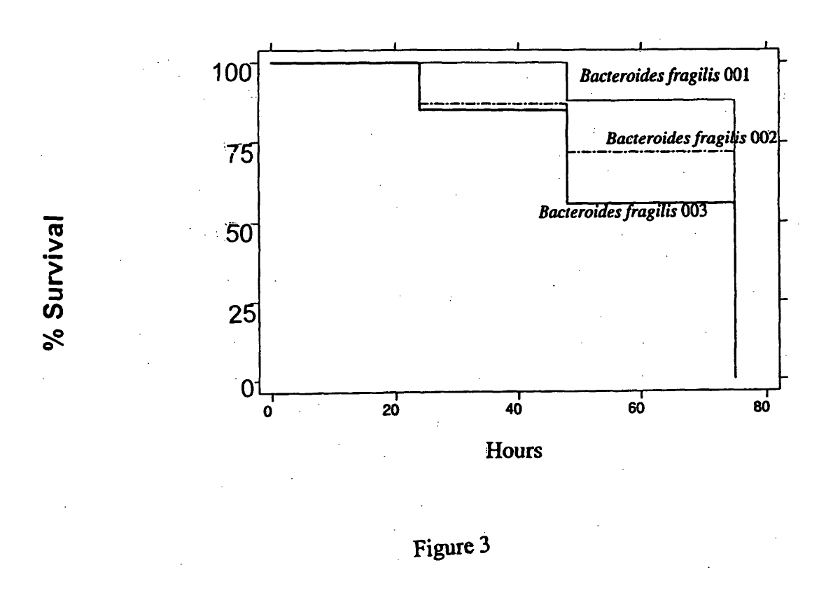

FIG. 3 shows the Kaplan-Meier survival estimates of killing of C. elegans, under low oxygen conditions, by three different strains of Bacteroides fragilis, 001, 002, and 003, as compared to E. coli OP-50.

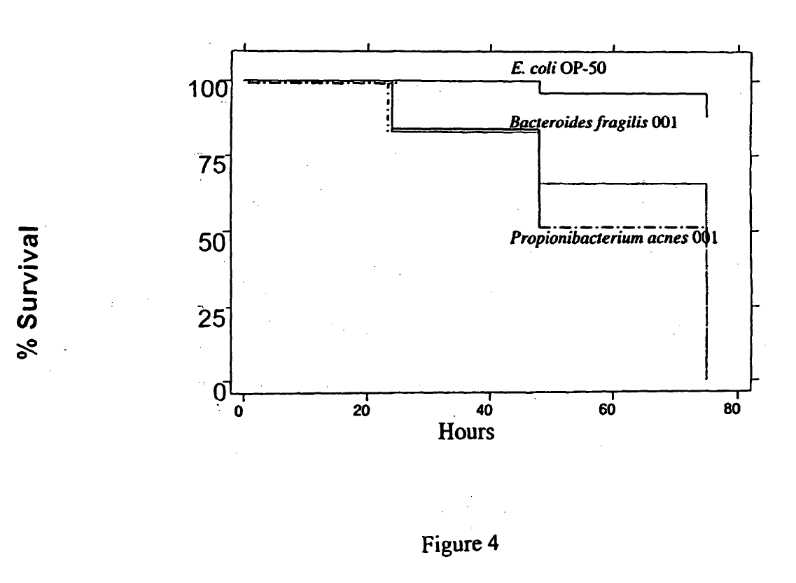

FIG. 4 shows the Kaplan-Meier survival estimates of killing of C. elegans, under low oxygen conditions, by Bacteroides fragilis strain 001 and Propionibacterium acnes, as compared to E. coli OP-50.

Below we describe experimental evidence demonstrating that several bacterial pathogens, including Enterococcus, Bacteroides, and Propionibacterium, cause disease in the nematode C. elegans under conditions of low oxygen, and that C. elegans feeding on lawns of these pathogens die over the course of a few days or within a few hours as a result of a pathogenic process. These low oxygen condition killing assays described herein provide a useful system for identifying novel virulence factors, as well as for identifying compounds that either inhibit pathogenicity, promote a host's resistance to a pathogen, or both. The following experimental examples are intended to illustrate, not limit, the scope of the claimed invention.

C. elegans Killing By Enterococcus Under Low Oxygen Conditions

To monitor Enterococcus faecium-mediated killing under low oxygen conditions, C. elegans assays were carried out as follows. Brain heart infusion (BHI) agar medium (Difco) was autoclaved and poured into 35 mm tissue culture plates (Fisher). Appropriate antibiotics were added to the medium before pouring that prevented growth of E. coli, but allowed growth of the particular Enterococcus faecium strain being tested. For strains A, B, and C, 50 μg/ml gentamicin was used.

Three different strains of Enterococcus faecium were obtained from the Microbiology Laboratory at the Massachusetts General Hospital, Boston, Mass. and were designated A, B, and C. In general, Enterococcus is considered the second most common organism recovered from nosocomial urinary tract and wound infections and the third most common cause of nosocomial bacteremia in the United States, and about 15-30% of these infections are due to Enterococcus faecium (Murray, Am. J. Med. 102:284-93, 1997).

Bacterial lawns of Enterococcus faecium were prepared as follows. On the tissue culture plate, 4 ml of fluid BHI broth (Difco) was inoculated with a single colony of the appropriate strain, grown under aerobic conditions (i.e, standard atmospheric conditions), at 37° C. for 12-24 hours, and 10 μl of the culture was then plated on each plate. The plates were incubated at 37° C. overnight under conditions of low oxygen, and then brought to room temperature for 2 to 5 hours (again under conditions of low oxygen). Low oxygen conditions were generated using the GasPak® or the GasPak Plus® System in conjunction with the BBL® Gas Generator Envelopes (Becton Dickinson, Sparks, Md. 21152) according to methods described by Brewer (Science 147:1033-1034, 1965), Brewer et al. (Appl. Microbiol. 14:135-136, 1966), and Seip et al. (J. Clin. Microbiol. 11:226-233, 1980).

Thirty C. elegans, at the L4 larval stage, cultured on a plate of OP50 E. coli, were then placed on a lawn of E. faecium. The plates were incubated at 25° C. under aerobic conditions and the number of worms found dead compared to the total number of plated worms was counted at approximately 20-minute intervals. Each experimental condition was performed in triplicate and repeated at least twice. The number of dead worms was calculated from results obtained from three plates for each strain. The data were then analyzed using Kaplan-Meier survival curves using statistical software from STATA Corporation (Stata Statistical Software: Release 6.0. College Station, Tex.: Stata Corporation, 1999).

Using the above-described killing assay protocol, the percentage of C. elegans dead as a function of time feeding on each of the Enterococcus faecium strain, under low oxygen, was determined (FIG. 1). Under completely aerobic conditions (i.e., when a lawn of Enterococcus faecium is grown without exposure to conditions of low oxygen) no killing of C. elegans was observed (FIG. 2). These results show that Enterococcus faecium is pathogenic to C. elegans when Enterococcus is grown under low oxygen conditions.

C. elegeans Killing By Bacteroides fragilis Under Low Oxygen Conditions

To monitor Bacteroides fragilis-mediated killing under low oxygen conditions, C. elegans assays were carried out as follows. Brain heart infusion (BHI) agar medium (Difco) was autoclaved and poured into 35 mm tissue culture plates (Fisher).

Three different strains of Bacteroides fragilis were obtained from the Microbiology Laboratory at the Massachusetts General Hospital, Boston, Mass. and were designated 001, 002, and 003. In general, Bacteroides fragilis is present in 75% of postoperative wound infections and anaerobic bacteria were found in 88 out of 112 specimens from children with ruptured appendicitis (Duerden BI CID 1994; Brook, Ann Surg. 1980:192:208-12,1980). Anaerobes account for at least 9% of bacteremias (or approximately one case per 1,000 admissions). Species from the Bacteroides fragilis group account for about 55% of anaerobic bacteremias, with a mortality of 19% and a 16-day increase in hospital stay (Goldstein, Clin. Infect. Dis. 23 (Suppl 1):S97-101, 1996).

Bacterial lawns of Bacteroides fragilis were prepared as follows. Five milliliters of THIO broth (Binax, Waterville, Me.) was inoculated with a single colony of the appropriate strain, grown at 37° C. for 12-24 hours, and 10 μl of the culture was then plated on each BHI plate. The plates were incubated at 37° C. overnight under conditions of low oxygen, and then brought to room temperature for 2 to 5 hours (again under conditions of low oxygen). A low oxygen condition was created using the GasPak® or GasPak Plus® System in conjunction with the BBL® Gas Generator Envelopes (Becton Dickinson, Sparks, Md.) according to methods described by Brewer (Science 147: 1033-1034, 1965), Brewer et al. (Appl. Microbiol. 14: 135-136, 1966), and Seip et al. (J. Clin. Microbiol. 11:226-233,1980).

Thirty C. elegans, at the L4 larval stage, were then placed on the lawn from a plate of OP50 E. coli. The plates were incubated at 25° C. under conditions of low oxygen as described herein and the number of worms found dead compared to the total number of plated worms was counted at approximately 24-hour intervals. Each experimental condition in the following experiments was done in triplicate and repeated at least twice. The number of dead worms was calculated from results obtained from three plates for each strain. The data were then analyzed using Kaplan-Meier survival curves using standard statistical software from StataCorp (Stata Statistical Software: Release 6.0. College Station, Tex.: Stata Corporation, 1999).

Using the above-described killing assay protocol, the percentage of C. elegans dead as a function of time feeding on each of the Bacteroides fragilis strains was determined (FIG. 3). These results show that Bacteroides fragilis is pathogenic to C. elegans under low oxygen conditions.

C. elegans Killing By Propionibacterium Under Low Oxygen

To monitor Propionibacterium acnes-mediated killing, C. elegans assays were carried out as follows. Brain heart infusion (BHI) agar medium (Difco) was autoclaved and poured into 35 mm tissue culture plates (Fisher).

Strains of Propionibacterium acnes were obtained from the Microbiology Laboratory at the Massachusetts General Hospital, Boston, Mass.

Bacterial lawns of Propionibacterium acnes were prepared as follows. On a tissue culture plate, 5 ml of fluid THIO broth (Binax, Waterville, Me.) was inoculated with a single colony of the appropriate strain, grown at 37° C. for 12-24 hours, and 10 μl of the culture was plated on each plate. The plates were incubated at 37° C. overnight under conditions of low oxygen, and then brought to room temperature for 2 to 5 hours (again under conditions of low oxygen). Low oxygen conditions were generated using the GasPak® or the GasPak Plus® System in conjunction with the BBL® Gas Generator Envelopes (Becton Dickinson, Sparks, Md., 21152) according to methods described by Brewer (Science 147: 1033-1034, 1965), Brewer et al. (Appl. Microbiol. 14: 135-136, 1966), and Seip (J. Clin. Microbiol. 11:226-233,1980).

Thirty C. elegans, at the L4 larval stage, were then placed on the lawn from a plate of OP50 E. coli. The plates were incubated at 25° C. under conditions of low oxygen and the number of worms found dead compared to the total number of plated worms was counted at approximately 30-minute intervals. Each experimental condition in the following experiments was done in triplicate and repeated at least twice. The number of dead worms was calculated from results obtained from three plates for each strain. The data were then analyzed using Kaplan-Meier survival curves using standard statistical software from STATA Corporation (Stata Statistical Software: Release 6.0. College Station, Tex.: Stata Corporation, 1999).

Using the above-described killing assay protocol, the percentage of C. elegans dead as a function of time feeding on each of the Propionibacterium acnes strains was determined (FIG. 4). These results show that Propionibacterium acnes is pathogenic to C. elegans under low oxygen conditions.

C. elegans Killing by Other Bacteria under Low Oxygen

Killing curves similar to the above-described curves have also been obtained using the following bacteria: Fusobacterium necrophorum, Fusobacterium nucleatum, Actinomyces israelli, Bacteroides ovatus, Bacteroides ureolyticus, and Lactobacillus casei.

Identifying Virulence Factors Expressed under Low Oxygen Conditions

Based on the results described above showing that microorganisms are virulent on C. elegans when they are under a low oxygen condition (Bacteroides and Propionibacterium), or when microorganisms are grown under a low oxygen condition (Enterococcus), we have developed a method for identifying virulence determinants important for pathogenicity of a pathogen under such conditions. The screen, in general, utilizes the above-described low oxygen/nematode killing assays and exploits the ability to readily screen thousands of randomly generated pathogen mutants. In addition to using wild type host worms in the killing assays, mutants that are constipated or defecation defective, such as aex-2 and unc-25, mutants that are grinding defective, such as phm-2 and eat-14, and specific ABC transporter mutants such as pgp-4 and mrp-1 may be utilized as well.

Exemplary pathogenic bacteria useful in the methods of the invention, include, without limitation, Aerobacter, Aeromonas, Acinetobacter, Agrobacterium, Arcobacter, Bacillus, Bacteroides, Bartonella, Biofidobacterium, Bilophilla, Bordetella, Bortella, Borrelia, Brucella, Burkholderia, Calymmatobacterium, Campylobacter, Citrobacter, Clostridium, Cornyebacterium, Enterobacter, Enterococcus, Escherichia, Eubacterium, Francisella, Fusobacterium, Gardnerella, Haemophilus, Hafnia, Helicobacter, Klebsiella, Legionella, Leptospira, Listeria, Mobiluncus, Morganella, Moraxella, Mycobacterium, Mycoplasma Neisseria, Nocardia, Pasteurella, Peptostreptococcus, Porphyromonas, Prevotella, Propionibacterium, Proteus, Providencia, Pseudomonas, Salmonella, Serratia, Shigella, Staphylococcus, Streptococcus, Stentorophomonas, Sutterella, Treponema, Xanthomonas, Veillonella, Vibrio, and Yersinia.

In general, a strain of a pathogen (for example, the bacterium Enterococcus) is mutated according to standard methods known in the art and then subsequently evaluated for its ability to induce disease in the nematode host organism. A mutagenized pathogen found to have diminished pathogenicity or which is rendered non-pathogenic is useful in the method of the invention. Such mutant pathogens are then used for identifying host-dependent or host-independent virulence factors responsible for pathogenicity according to methods known in the art.

With respect to producing a low oxygen condition, one of skill in the art will recognize that a variety of methods are known for culturing nematodes (see, for example, Honda et al., Journal of Gerontology 48:B57-B61, 1993) and pathogens under such conditions. In addition, apparatuses and methods for generating a low oxygen atmosphere are described, for example, in Stoermer et al. (U.S. Pat. No. 4,976,931). For example, plates, containing nematodes infected with a pathogen such as Enteroccocus facecium, are incubated in a BBL® GasPak® anaerobic jar (Becton Dickinson, Sparks, Md.). Methods for culturing pathogens under low oxygen conditions are also known in the art, including the culturing of bacterial pathogens in commercially available broth or blood agar (Difco Laboratories, Detroit, Mich.) under low oxygen conditions. Moreover, other methods are also available for creating conditions of low oxygen for culturing, such methods include the use of an anaerobic chamber (for example, a large, closed chamber that maintains an anaerobic condition); a candle jar (for example, a jar with a candle in it that can consume oxygen, creating an anaerobic condition); or an airtight plastic chamber in which oxygen or nitrogen is displaced using standard methods to produce conditions of less oxygen.

Furthermore, one skilled in the art recognizes that exposure of the pathogen, nematode, or both to a low oxygen environment may occur independently, sequentially, or continuously.

The following is a working example of a virulence factor nematode screening system which utilizes the human clinical isolate Bacteroides fragilis found to be infectious in the above-described C. elegans nematode feeding model. The advantage of using a nematode as a host for studying this mammalian pathogen is the relative simplicity of identifying non-pathogenic Bacteroides mutants in the nematode.

In one preferred working example, in which survival is monitored, four to eight C. elegans worms (e.g., L4 larvae) are placed on a lawn of mutagenized Bacteroides spp., and survival is monitored after approximately forty to eighty hours according to the methods described herein. A Bacteroides pathogen, such as Bacteroides fragilis, is mutated according to any standard procedure, e.g., standard in vivo or in vitro insertional/transponson mutagenesis methods (see, e.g., Ike et al., J. Bacteriol. 172:155-63, 1990; Munkenbeck et al., Plasmid 24: 57-67, 1990; Kleckner et al., J. Mol. Biol. 116: 125, 1977). Other methods are also available, e.g., chemical mutagenesis, or directed mutagenesis of DNA. After approximately forty to eighty hours, very few or no live worms are found on a plate seeded with wild-type, pathogenic Bacteroides fragilis, whereas on a plate with mutagenized Bacteroides fragilis, increased survival (e.g., as determined by an increased LT₅₀) of the worms is observed. Thus, the ability of worms to grow in the presence of mutated Bacteroides fragilis is an indication that a gene responsible for pathogenicity has been inactivated. The positions of the inactivating mutations are then identified using standard methods, (e.g., by polymerase chain reaction and sequencing of insertion/transposon junctions or by mapping), leading to the cloning and identification of the mutated virulence factor(s) (e.g., by nucleotide sequencing).

In another working example, in which survival and reproduction is monitored, four to eight C. elegans worms (e.g., L4 hermaphrodite larvae) are placed on a lawn of mutagenized Enterococcus faecium, and worm progeny is monitored. Enterococcus faecium is mutated according to standard methods. After approximately one hundred to one hundred fifty minutes, very few or no live worms are found on a plate seeded with wild-type, pathogenic E. faecium, whereas on a plate with the E. faecium mutant, hundreds or thousands of live progeny of the initial two hermaphrodite worms are present. Thus, the ability of worms to grow and reproduce in the presence of mutated E. faecium is taken as an indication that a gene responsible for pathogenicity has been inactivated. The mutated virulence factor is then identified using standard methods.

In another preferred working example, in which survival is monitored, four to eight C. elegans worms (e.g., L4 larvae) are placed on a lawn of mutagenized Clostridium spp. or on a lawn of Clostridium spp. spores. Standard methods for the culture of Clostridium are known to the skilled artisan (Peck et al., Int. J. Food Microbiol. 28:289-97, 1995; Kihm et al., Appl. Environ. Microbiol. 56:681-5, 1990). C. elegans survival is monitored every twenty-four hours according to methods described herein. A Clostridium pathogen, such as Clostridium botulinum, is mutated according to any standard procedure, e.g., standard in vivo or in vitro insertional/transponson mutagenesis methods (see, e.g., Ike et al., J. Bacteriol. 172:155-63, 1990; Munkenbeck et al., Plasmid 24: 57-67, 1990; Kleckner et al., J. Mol. Biol. 116: 125, 1977). Other methods are also available, e.g., chemical mutagenesis, or directed mutagenesis of DNA. After approximately forty to eighty hours, very few or no live worms are found on a plate seeded with wild-type, pathogenic Clostridium botulinum or Clostridium botulinum spores, whereas on a plate with mutagenized Clostridium botulinum or Clostridium botulinum spores, increased survival (e.g., as determined by an increased LT50) of the worms is observed. Thus, the ability of worms to grow in the presence of mutated Clostridium botulinum is an indication that a gene responsible for pathogenicity has been inactivated. The positions of the inactivating mutations are then identified using standard methods, (e.g., by polymerase chain reaction and sequencing of insertion/transposon junctions or by mapping), leading to the cloning and identification of the mutated virulence factor(s) (e.g., by nucleotide sequencing).

Mouse Pathogenicity Screening Assays

To further evaluate the virulence of mutants identified in the above-described nematode screening assays, mouse pathogenicity/mortality studies are performed according to standard methods. Female ICR Mice (Taconic, Germantown, N.Y. or Charles River, Wilmington, Mass.) weighing 20 to 30 grams and housed 5 per cage, are used for evaluating the virulence of the mutants. Mice, in groups of 6-10, are injected intraperitoneally with mutant bacteria, for example, Enterococcus faecium or Clostridium botulinum using standard methods. For Enterococcus faecium, for example, this injection may be in a sterile rat fecal extracts (SRFE) as described below. The survival of mice receiving mutant bacteria is then compared to the survival of animals receiving an equal inoculum of wild-type bacteria (e.g., without a mutation). All animals have access to chow and water ad libitum throughout an experiment.

An exemplary bacterial inoculum is prepared as follows. Enterococcus faecium or Clostridium botulinum, or Enterococcal or Clostridium mutants, are grown overnight in BHI broth at 37° C. with gentle shaking. The cells are harvested by centrifugation, washed once with 0.9% saline, and then are resuspended in saline to an optical density of 1.6 to 2.2 at 600 nm. CFUs (colony-forming units) of cells suspensions are determined by plating serial dilutions onto BHI agar plates. Serial dilutions are prepared in saline and mixed with SRFE, or other suitable medium, to the desired inoculum. For the preparation of SRFE, rat feces are dried, crushed, mixed with a volume of sterile distilled water three times that of the feces, and autoclaved. The resultant slurry is centrifuged, and the fecal extracts are removed aseptically. The extracts are then autoclaved and mixed with an enterococcal culture. Each inoculum is then diluted to a final 35% SRFE to yield the desired final inoculum.

Using a 25-gauge needle, mice are injected intraperitoneally with a 1 ml inoculum containing approximately 5×10⁸ to 1×10⁹ colony forming units of Enterococcus faecium or Clostridium botulinum, or Enterococcal or Clostridium mutants. After injection the animals are returned to their cages and monitored every 8 hours for seven days. Surviving animals are then sacrificed and examined by autopsy. Control mice injected intraperitoneally with 1 ml of sterile SRFE are also examined.

Upon autopsy, bacteria are recovered from the kidneys or spleens under aseptic conditions. Peritoneal fluid and abdominal abscesses are also sampled for evaluation. Serial dilutions of the peritoneal fluid are prepared and 0.1 ml of each dilution is spread on agar plates for colony counts. Plates are then incubated under aerobic (or other appropriate culture conditions, such as low oxygen) for up to 4 days. BHI plates containing rifampin (for culturing Enterococcus faecium) or another appropriate antibiotic (for culturing Clostridium botulinum) or rifampin and erythromycin (for culturing Enterococcal mutants) or another appropriate combination of antibiotics (for culturing Clostridium mutants) are used for selection. Results are expressed, for example, by Kaplan-Meier curves and log rank test using STATA software (StataCorp. 1999. Stata Statistical Software: Release 6.0. College Station, Tex.: Stata Corporation).

Mutants showing a statistically significant difference or a statistical trend (P≧0.20) compared to the wild type are, if desired, evaluated a second time. Mutants identified as having reduced virulence are taken as being useful in the invention.

Compound Screening Assays

As discussed above, our experimental results demonstrated that bacterial virulence factors are involved in pathogenicity of the nematode, C. elegans under a low oxygen condition. Based on this discovery we have also developed a screening procedure for identifying therapeutic compounds (e.g., anti-pathogenicity pharmaceuticals) which can be used to inhibit the ability of a pathogen to cause infection under a low oxygen condition. In general, the method involves screening any number of compounds for therapeutically-active agents by employing the low oxygen/nematode killing system described herein. Based on our demonstration that several bacterial pathogens infect and kill C. elegans under such conditions, it will be readily understood that a compound that interferes with the pathogenicity of a pathogen in a nematode also provides an effective therapeutic agent in a mammal (e.g., a human patient). Whereas most antibiotics currently in medical use are either bactericidal or bacteriostatic, thus favoring resistant strains or mutants, the compounds identified in the screening procedures described herein do not kill the bacteria but instead render them non-pathogenic. Moreover, since the screening procedures of the invention are performed in vivo, it is also unlikely that the identified compounds will be highly toxic to the host organism.

Accordingly, the methods of the invention simplify the evaluation, identification, and development of active therapeutic agents such as drugs for the treatment of pathogenic diseases caused by a pathogen.

In general, the chemical screening methods of the invention provide a straightforward means for selecting natural product extracts or compounds of interest from a large population which are further evaluated and condensed to a few active and selective materials. Constituents of this pool are then purified and evaluated in the methods of the invention to determine their anti-pathogenic activity.

Test Extracts and Compounds

In general, novel anti-pathogenic drugs are identified from large libraries of both natural product or synthetic (or semi-synthetic) extracts or chemical libraries according to methods known in the art. The screening method of the present invention is appropriate and useful for testing compounds from a variety of sources for possible anti-pathogenic activity. The initial screens may be performed using a diverse library of compounds, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

For example, those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have anti-pathogenic activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for the treatment of pathogenicity are chemically modified according to methods known in the art.

Since many of the compounds in libraries such as combinatorial and natural products libraries, as well as in natural products preparations, are not characterized, the screening methods of this invention provide novel compounds which are active as inhibitors or inducers in the particular screens, in addition to identifying known compounds which are active in the screens. Therefore, this invention includes such novel compounds, as well as the use of both novel and known compounds in pharmaceutical compositions and methods of treating.

Exemplary High Throughput Screening Systems

To evaluate the efficacy of a molecule or compound, under a low oxygen condition, in promoting host resistance to, or inhibiting pathogenicity of a pathogen, for example, any of the above-described bacteria or spores, a number of high throughput assays may be utilized.

For example, to enable mass screening of large quantities of natural products, extracts, or compounds in an efficient and systematic fashion, Caenorhabditis elegans, (e.g., L4 hermaphrodite larvae or a mutant worm such as aex-2, unc-25, phm-2, eat-14, pgp-4, or mrp-1), are cultured in wells of a microtiter plate, facilitating the semiautomation of manipulations and full automation of data collection. As is discussed above, E. faecium, cultured in a low oxygen environment, infects and kills C. elegans. If E. faecium has diminished pathogenicity, then L4 worms live, develop into adult hermaphrodites, and produce thousands of live progeny. Accordingly, if C. elegans is incubated with the pathogen, the worms will die, unless a compound is present to reduce E. faecium pathogenicity. The presence of such live progeny is easily detected using a variety of methods, including visual screening with standard microscopes.

To evaluate the ability of a test compound or extract to promote a host's resistance to a pathogen or to repress pathogenicity of a pathogen, a test compound or extract is inoculated at an appropriate dosage into an appropriate agar medium (e.g., BHI or M17 (Difco)) seeded with an appropriate amount of an overnight culture of a pathogen, e.g., E. faecium cultured under low oxygen conditions as described herein. If desired, various concentrations of the test compound or extract can be inoculated to assess dosage effect on both the host and the pathogen. Control wells are inoculated with non-pathogenic bacteria (negative control) or a pathogen in the absence of a test compound or extract (positive control). Plates are then incubated 24 hours under low oxygen conditions at 37° C. to facilitate the growth of the pathogen. Microtiter dishes are subsequently cooled to 25° C., and two C. elegans L4 hermaphrodite larva are added to the plate and incubated at 25° C., the upper limit for normal physiological integrity of C. elegans. At an appropriate time interval, e.g., one hundred to two hundred minutes, wells are examined for surviving worms, the presence of progeny, or both, e.g., by visual screening or monitoring motion of worms using a motion detector.

In another working example, Bacteroides-mediated killing of C. elegans, under a low oxygen condition, is carried out as follows. Brain heart infusion (BHI) agar medium (Difco) is autoclaved and poured into 35 mm tissue culture plates (Fisher). Appropriate antibiotics are added to the medium before pouring to prevent growth of E. coli, but allow for the growth of the particular Bacteroides strains being tested. A test compound or compound library is also added to the medium. On the tissue culture plate, 2 ml of BHI is inoculated with a single colony of the appropriate strain, grown at 37° C. for 4 to 5 hours, and 10 μl of the culture is plated on each plate. The plates are incubated at 37° C. overnight, and then brought to room temperature for 2 to 5 hours, also under low oxygen conditions. Thirty C. elegans, at the L4 larval stage, are then placed on the lawn from a plate of OP50 E. coli. The plates are incubated at 25° C. under low oxygen conditions as described herein and the number of worms found dead compared to the total number of plated worms are then counted at approximately 24 hour intervals. Each experimental condition is done in triplicate and repeated at least twice. At an appropriate time interval plates are examined for surviving worms.

Comparative studies between treated and control worms (or larvae) are used to determine the relative efficacy of the test molecule or compound in promoting the host's resistance to the pathogen or inhibiting the virulence of the pathogen. A test compound which effectively stimulates, boosts, enhances, increases, or promotes the host's resistance to the pathogen or which inhibits, inactivates, suppresses, represses, or controls pathogenicity of the pathogen, and does not significantly adversely affect the normal physiology, reproduction, or development of the worms is considered useful in the invention.

In another working example, Clostridum-mediated killing of C. elegans, under a low oxygen condition, is carried out as follows. Brain heart infusion (BHI) agar medium (Difco) is autoclaved and poured into 35 mm tissue culture plates (Fisher). Appropriate antibiotics are added to the medium before pouring to prevent growth of E. coli, but allow for the growth of the particular Clostridium strains being tested. A test compound or compound library is also added to the medium. On the tissue culture plate, 2 ml of BHI is inoculated with a single colony of the appropriate strain, grown at 37° C. for 4 to 5 hours, and 10 μl of the culture is plated on each plate. The plates are incubated at 37° C. overnight, and then brought to room temperature for 2 to 5 hours, also under low oxygen conditions. Thirty C. elegans, at the L4 larval stage, are then placed on the lawn from a plate of OP50 E. coli. The plates are incubated at 25° C. under low oxygen conditions as described herein and the number of worms found dead compared to the total number of plated worms are then counted at twenty-four hour intervals. Each experimental condition is done in triplicate and repeated at least twice.

Comparative studies between treated and control worms (or larvae) are used to determine the relative efficacy of the test molecule or compound in promoting the host's resistance to the pathogen or inhibiting the virulence of the pathogen. A test compound which effectively stimulates, boosts, enhances, increases, or promotes the host's resistance to the pathogen or which inhibits, inactivates, suppresses, represses, or controls pathogenicity of the pathogen, and does not significantly adversely affect the normal physiology, reproduction, or development of the worms is considered useful in the invention.

Use

The methods of the invention provide a simple means for identifying virulence factors expressed under low oxygen and compounds capable of either inhibiting pathogenicity or enhancing an organism's resistance capabilities to such pathogens. Accordingly, a chemical entity discovered to have medicinal value using the methods described herein are useful as either drugs, or as information for structural modification of existing anti-pathogenic compounds, e.g., by rational drug design.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections which provide continuous, sustained levels of the drug in the patient. Treatment of human patients or other animals will be carried out using a therapeutically effective amount of an anti-pathogenic agent in a physiologically-acceptable carrier. In the context of treating a bacterial infection a “therapeutically effective amount” or “pharmaceutically effective amount” indicates an amount of an antibacterial agent, e.g., as disclosed for this invention, which has a therapeutic effect. This generally refers to the inhibition, to some extent, of the normal cellular functioning of bacterial cells causing or contributing to a bacterial infection. The dose of antibacterial agent which is useful as a treatment is a “therapeutically effective amount.” Thus, as used herein, a therapeutically effective amount means an amount of an antibacterial agent which produces the desired therapeutic effect as judged by clinical trial results, standard animal models of infection, or both. This amount can be routinely determined by one skilled in the art and will vary depending upon several factors, such as the particular bacterial strain involved and the particular antibacterial agent used. This amount can further depend on the patient's height, weight, sex, age, and renal and liver function or other medical history. For these purposes, a therapeutic effect is one which relieves to some extent one or more of the symptoms of the infection and includes curing an infection.

The compositions containing antibacterial agents of virulence factors or genes can be administered for prophylactic or therapeutic treatments, or both. In therapeutic applications, the compositions are administered to a patient already suffering from an infection from bacteria (similarly for infections by other microbes), in an amount sufficient to cure or at least partially arrest the symptoms of the infection. An amount adequate to accomplish this is defined as “therapeutically effective amount.” Amounts effective for this use will depend on the severity and course of the infection, previous therapy, the patient's health status and response to the drugs, and the judgment of the treating physician. In prophylactic applications, compositions containing the compounds of the invention are administered to a patient susceptible to, or otherwise at risk of, a particular infection. Such an amount is defined to be a “prophylactically effective amount.” In this use, the precise amounts again depend on the patient's state of health, weight, and the like. However, generally, a suitable effective dose will be in the range of 0.1 to 10000 milligrams (mg) per recipient per day, preferably in the range of 10-5000 mg per day. The desired dosage is preferably presented in one, two, three, four, or more subdoses administered at appropriate intervals throughout the day. These subdoses can be administered as unit dosage forms, for example, containing 5 to 1000 mg, preferably 10 to 100 mg of active ingredient per unit dosage form. Preferably, the compounds of the invention will be administered in amounts of between about 2.0 mg/kg to 25 mg/kg of patient body weight, between about one to four times per day.

Suitable carriers and their formulation are described, for example, in Remington's Pharmaceutical Sciences by E. W. Martin. The amount of the anti-pathogenic agent to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation.

All publications and patents mentioned in this specification are herein incorporated by reference to the same extent as if each individual publication or patent was specifically and individually incorporated by reference.

From the foregoing description, one skilled in the art can easily ascertain the essential characteristics of this invention; can make various changes and modifications of the invention to adapt it to various usages and conditions. Thus, other embodiments are also within the claims.

1. A method for identifying a pathogenic virulence factor, comprising the steps of:
(a) exposing a nematode to a mutagenized pathogen cultured under a low oxygen condition;
(b) determining whether said mutant pathogen infects said nematode, a reduction of disease in said nematode relative to that caused by the non-mutagenized pathogen indicating a mutation in a pathogenic virulence factor; and
(c) using said mutation as a marker for identifying said pathogenic virulence factor.

2. The method of claim 1, wherein said nematode and said mutant pathogen are cultured together under a low oxygen condition.

3. The method of claim 1, wherein said pathogen is a bacterium.

4. The method of claim 1, wherein said pathogen is in the form of a spore.

5. The method of claim 1, wherein said pathogen is a member of the genus of Enterococcus.

6. The method of claim 1, wherein said pathogen is a member of the genus of Bacteroides.

7. The method of claim 1, wherein said pathogen is a member of the genus of Propionibacterium.

8. The method of claim 1, wherein said pathogen is a member of the genus of Clostridium.

9. The method of claim 1, wherein said nematode is Caenorhabditis elegans.

10. The method of claim 1, wherein said method utilizes a bacterial/C. elegans killing assay.

11. The method of claim 10, wherein said bacterial pathogen causes less C. elegans killing than the non-mutagenized bacterial pathogen.

12. A method of identifying a compound that inhibits pathogenicity of a bacterial pathogen, comprising the steps of:
(a) providing a nematode comprising a pathogen cultured under a low oxygen condition;
(b) contacting said nematode with a test compound; and
(c) determining whether the test compound inhibits the pathogenicity of said pathogen in said nematode.

13. The method of claim 12, wherein said nematode and said mutant pathogen are cultured together under a low oxygen condition.

14. The method of claim 12, wherein said pathogen is a bacterium.

15. The method of claim 12, wherein said pathogen is in the form of a spore.

16. The method of claim 12, wherein said pathogen is a bacterium belonging to the genus of Enterococcus.

17. The method of claim 12, wherein said pathogen is a bacterium belonging to the genus of Bacteroides.

18. The method of claim 12, wherein said pathogen is a member of the genus of Bacteroides.

19. The method of claim 12, wherein said pathogen is a member of the genus of Clostridium.

20. The method of claim 12, wherein said nematode is Caenorhabditis elegans.

21. The method of claim 12, wherein said test compound is provided in a compound library.

22. The method of claim 12, wherein said test compound is a small organic compound.

23. The method of claim 12, wherein said test compound is a peptide, peptidomimetic, or antibody or fragment thereof.

24. The method of claim 12, wherein said inhibition of pathogenicity is measured by a bacteria/C. elegans killing assay.

25. The method of claim 24, wherein said bacterial pathogen causes less C. elegans killing in the presence of said test compound than in the absence of said test compound.
